(19) United States
(12) Patent Application Publication
(10) Pub. No.: US 2019/0117669 A1
(43) Pub. Date: Apr. 25, 2019

(54) Methods for Treating NSAID-Induced Cardiovascular, Cerebrovascular, or Renovascular Adverse Events

(71) Applicant: University of Utah Research Foundation, Salt Lake City, UT (US)

(72) Inventor: Mark A. Munger, Sandy, UT (US)

(73) Assignee: University of Utah Research Foundation, Salt Lake City, UT (US)

(21) Appl. No.: 16/092,501

(22) PCT Filed: Mar. 3, 2017
(86) PCT No.: PCT/US2017/020611

§ 371 Date: Oct. 10, 2018

Related U.S. Application Data
(60) Provisional application No. 62/320,857, filed on Apr. 11, 2016.

(51) Int. Cl. A61K 31/5575; A61K 9/28; A61K 9/20; A61P 29/02; A61P 1/04

(52) U.S. Cl. CPC A61K 31/5575; A61K 9/28; A61K 45/06; A61P 29/02; A61P 1/04; A61K 9/2072

(57) ABSTRACT

Methods and compositions for reducing the risk of cardiovascular, cerebrovascular, or renovascular adverse events are provided. Generally, the methods include administering to a subject taking an NSAID a therapeutically effective amount of a misoprostol compound. The methods can also include administering to a subject in need thereof a therapeutically effective amount of an NSAID and therapeutically effective amount of a misoprostol compound.

### CROSS REFERENCES TO RELATED APPLICATIONS

This application claims priority to U.S. Provisional Application No. 62/320,857, filed Apr. 11, 2016, the entire contents of which are incorporated by reference herewith.

### FIELD OF THE INVENTION

The present disclosure relates to methods and compositions for reducing the risk of NSAID-induced cardiovascular, cerebrovascular, and/or renovascular adverse events.

### BACKGROUND OF THE INVENTION

Nonsteroidal anti-inflammatory drugs (NSAIDs) are nonselective inhibitors of the enzyme cyclooxygenase (COX) inhibiting cyclooxygenase 1 (COX 1) and cyclooxygenase 2 (COX-2) isoenzymes.¹ COX catalyzes the formation of prostaglandins and thromboxane from arachidonic acid. The COX enzyme catalyzes the formation of prostacyclins and thromboxane from arachidonic acid. COX-1 is a constitutively expressed enzyme regulating many physiological functions through consistent enzymatic production of prostaglandins including maintenance of the gastrointestinal lining, by inhibiting acid secretion from parietal cells in the stomach, paracrine or autocrine vasodilation, inhibition of blood platelet activity, regulation of smooth muscle tissue, inflammation, and glomerular filtration rate. COX-2 activity is induced by pro-inflammatory cytokines which lead to immune responses such as inflammation.

NSAIDs are among the most commonly used medications worldwide.²⁻³ The common use of NSAIDs results from indications for the symptomatic relief of acute and chronic conditions producing associated pain and inflammation. The use of NSAIDs also comes with a broad range of adverse effects, including gastrointestinal, cardiovascular, cerebrovascular, or renovascular adverse events contributing to substantial morbidity and mortality.

NSAIDs are associated with a variety of gastrointestinal adverse effects, ranging from dyspepsia, heartburn, nausea and abdominal pain to more severe events such as gastrointestinal bleeding and ulcers. These gastrointestinal adverse effects are associated with two mechanisms: (1) direct local damage to the gastric mucosa primarily seen with acidic NSAIDs that are un-ionized (for example, ibuprofen, naproxen and ketoprofen) and (2) inhibition of cyclooxygenase with subsequent inhibition of synthesis of protective prostaglandins in the gastrointestinal mucosa.⁵ Evidence exists that gastrointestinal events from NSAIDs increase the risk of endoscopic to serious ulceration complications of 2.5-5 fold compared to persons not taking NSAIDs.⁶⁻⁸ Griffin and colleagues evaluated the risk of peptic ulcer disease associated with the use of NSAIDs in patients≥65 years old, finding a dose-related elevation of peptic ulcer disease with a relative risk of 2.8 (95% CI: 1.8-4.3) for the lowest dose to 8.0 (95% CI: 4.4-14.8) for the highest doses. There was a two-fold increase in gastrointestinal complications with NSAIDs. OTC doses of NSAIDs when taken at doses near the maximum recommended dosage had a ≥6-fold risk of gastrointestinal adverse events when compared to lower dosages recommended for OTC products.⁹

NSAIDs carry a substantial risk of cardiovascular adverse events.¹⁰⁻¹² This has resulted in the U.S. Food and Drug Administration (FDA) issuing two public health advisories for the voluntary removal of two COX-2 inhibitor drugs (rofecoxib and valdecoxib) from the U.S. market and most recently strengthening label warnings to state that NSAIDs increase the chance of a heart attack or stroke.¹³⁻¹⁵ The U.S. FDA recommends that patients and health care professionals should remain alert for heart-related side effects the entire time that NSAIDs are being taken. The risk of heart failure as a cardiovascular adverse event is also increased two-fold by NSAIDs.¹⁶⁻¹⁷

Several mechanisms contribute to an increased risk of NSAID-induced cardiovascular events from inhibition of COX-2 receptors including elevating blood pressure¹⁸, augmenting dietary sodium pressor response¹⁹, modulation of in-vivo vascular remodeling from hypertensive hemodynamic stress²⁰, and promoting a mismatch between COX-2 production of pro-aggregatory thromboxane effect on platelets and the anti-aggregatory sprostacyclin effects in endothelial cells.²¹⁻²² Deletion of the PGI2 receptor promotes early progression of atherosclerosis in mice genetically predisposed to hyperlipidemia and salt-sensitive hypertension.²³⁻²⁴ COX-2 inhibition is also the dominate source of PGE2 biosynthesis in humans under physiological conditions.^{18, 20} These mechanisms based on suppression of prostacyclins can lead over time to hypertension, progression of atherosclerosis and thrombosis. These laboratory findings support the clinical findings, being that the longer the NSAID exposure, incorporating dose, duration of action and treatment, the greater the likelihood of an adverse cardiovascular event.

Approximately 2.5 million persons experience a nephrotoxic event annually from taking NSAIDs.²⁵ An acute kidney injury can occur with the use of NSAIDs, with relative risks of hospitalization within 30 days from the initiation of NSAIDs of 1.5-2.4 (95% CI: 1.61-2.60), compared with unexposed persons.²⁵

Risk factors for NSAID-induced acute renovascular injury include NSAID higher doses²⁶, underlying chronic kidney disease²⁷, volume depletion from aggressive diuresis, vomiting or diarrhea, or heart failure.²⁸ Diuretics, angiotensin II enzyme inhibitors or angiotensin receptor blockers in combination with NSAIDs may increase the risk of NSAID-induced acute kidney injury (OR: 1.82, 95% CI: 1.35-2.46) (OR=Odds Ratio; CI=Confidence Interval). The highest risk is within the first 30 days of NSAID use.²⁶ Despite the known renal risks of NSAIDs, especially in patients with chronic kidney disease, the use of these agents continues with approximately 5% use in patients with an eGFR of 15-59 ml/min/1.73 m2.²⁹ Reno-vascular toxicity of NSAIDs is increased in a dose-dependent manner (OR: 0.94 [95% CI:0.58-1.51], 1.89 [95% CI: 1.34-2.67], and 2.32 [95% CI:1.45-3.71] at doses of ≤1200 mg, >1200 mg and ≥2400 mg/day).³⁰

Prostaglandins are critical to the functioning of the kidney. COX-1 and COX-2 are produced at the glomerular and vascular endothelium, the medullary and cortical collecting tubules, and medullary interstitial cells. Renal prostaglandins function as vasodilators in the kidneys. Under basal conditions, prostaglandins have no significant role in the regulation of renal perfusion, however, in the setting of reduced renal perfusion from vasoconstriction (e.g., angiotensin II, norepinephrine, vasopressin, endothelin) prostaglandin synthesis is increased to maintain renal perfusion and minimize ischemia.³¹⁻³⁴ Prostaglandins also increase renin secretion, are a counter-balance to vasopressin effects on water retention, and enhance sodium excretion.³⁴⁻³⁷ Prostaglandin synthesis is increased in the settings of prolonged renal vasoconstriction, serving to protect the glomerular filtration rate. NSAIDs inhibit prostaglandin-mediated afferent vasodilation and reduce peritubular blood flow raising the risk of ischemic acute tubular necrosis. Prostaglandins also cause sodium and water retention with increased systemic vascular resistance from neuro-hormonal activation contributing to hypertension and raising the risk of cardiovascular events.³⁸ Seniors are at increased risk kidney damage because of increased prostaglandin synthesis.

At a population level, all patients who ingest NSAIDs are at an elevated risk of experiencing potential cardiovascular, cerebrovascular, or renovascular adverse events. While one cannot predict whether any single individual will experience an adverse event with any degree of certainty, population studies have demonstrated that individuals with certain risk factors are at higher risk for having an NSAID-induced adverse event than those individuals missing the risk factors. The risk factors include but are not limited to: type 1 or type 2 diabetes mellitus, tobacco use (smoking or chewing), hypertension, hyperlipidemia, left ventricular hypertrophy, coronary artery disease, heart failure, peripheral vascular disease, cerebrovascular disease, renovascular disease, thromboembolic disease, physical inactivity, family history of such events, acute or chronic stress, diet high in saturated fat, social isolation, anxiety and depression, taking oral contraceptives or hormone replacement therapy, sleep apnea, atrial fibrillation, high alcohol consumption, obesity, ethnicity, allergy to prescription drugs, illicit drug use, ureter or kidney obstructions, lupus, and the use of contrast dyes for radiological tests.⁴⁵⁻⁴⁷

Data from the CDC's 2012 National Health and Nutrition Survey (NHANES) offers information on the number of people 18 and older in 2012 who suffered from the following diseases: intestinal ulcers (15,435,000), cardiovascular disease (234,921,000), stroke (6,370,000), and renal disease (3,882,000).⁴⁸ This provides perspective that the risk for a cardiovascular, cerebrovascular, or renovascular adverse event is much greater than the risk for a gastrointestinal adverse event in the population potentially exposed to NSAIDs.

Notwithstanding the larger risk for cardiovascular, cerebrovascular, or renovascular adverse events relative to gastrointestinal adverse events, pharmaceutical research and development has primarily focused on reducing the gastrointestinal adverse effects of NSAIDs. Several compounds have been developed to reduce the risk of gastrointestinal adverse effects of NSAIDs including nitric oxide donating NSAIDs, misoprostol (e.g. combined with diclofenac), histamine-2 receptor antagonists, and proton-pump inhibitors.

Misoprostol is a synthetic prostaglandin structurally related to prostaglandin E1 (PGE1).³⁹ There is high affinity of misoprostol for the prostaglandin receptor. Misoprostol is indicated for reducing the risk of NSAID-induced gastric ulcers in patients at high risk of complications from gastric ulcers, e.g., the elderly. See e.g. U.S. Pat. No. 5,601,843. A few studies have investigated the use of misoprostol for non-gastrintestinal adverse events, however, those studies have been regarded as unsuccessful and not generally accepted within the scientific community.^{41,42,44, 49} See also U.S. Pat. No. 8,552,059. In fact, the overwhelming consensus in the scientific literature establishes that misoprostol is not effective for non-gastrointestinal adverse events. For example:
1) Prostaglandin analogs do not improve renal function in rats receiving celecoxib 40 mg/kg) co-administered with misoprostol 100 mcg/kg) for 3-9 days. Blood pressure was increased in all misoprostol-treated groups.⁵⁹
2) In a human renal transplant and non-transplant meta-analysis, misoprostol did not demonstrate positive effects on renal outcomes (transplant renal transplant rejection or renal dysfunction 0.91 [95% CI: 0.64-1.28]), and no significant effect was found when comparing misoprostol versus placebo on non-transplant glomerular filtration rate mean differences 0.5 ml/min [95% CI:-2.8-1.8]).⁵¹
3) In a gentamicin-induced nephrotoxic dog model, misoprostol administered 3 mcg/kg every 8 hours for 8 days resulted in more severe azotemia, hyperphosphatemia, and renal histopathologic changes than for dogs administered gentamicin alone.⁵²
4) Misoprostol did not improve renal function in cirrhotic patients with and without ascites, was not protective in rheumatic arthritis patients receiving the nephrotoxic agent, cyclosporine A, and did not significantly alter renal parameters in patients with chronic renal insufficiency.53-56

Thus, to date, misoprostol has only been used in combination with NSAIDs to reduce gastrointestinal adverse effects.

While there have been attempts to reduce the risk of cardiovascular, cerebrovascular and renovascular adverse effects of NSAIDs, these attempts have not resulted in a compound that successfully reduces the risk of cardiovascular, cerebrovascular and renovascular adverse effects. See e.g. U.S. Pat. Nos. 9,173,427; 9,161,927; 9,114,068; 9,084,769, 9,090,566; 9,050,311; 8,940,891; 8,911,752; 8,901,159; 8,895,536; 8,624,002; 8,552,059; 8,242,146; 7,846,914; 7,754,772; 7,547,715; 7,432,107; 7,220,749; 6,790,864; 6,710,086; 6,710,086; 6,201,028; 5,955,451; and 5,252,602. Therefore, at present, there is no scientifically proven acceptable method of reducing the known risks of cardiovascular, cerebrovascular, renovascular events induced by NSAIDs. Therapies that reduce the risks of cardiovascular, cerebrovascular, renovascular events induced by NSAIDs are urgently needed.

### SUMMARY OF THE INVENTION

The present disclosure provides methods and compositions for reducing the risk of NSAID-induced cardiovascular, cerebrovascular, and/or renovascular adverse events. In certain embodiments, the methods of reducing NSAID-induced risk of cardiovascular disease, cerebrovascular disease, and/or renovascular disease comprise administering to a subject in need thereof a therapeutically effective amount of a NSAID and a NSAID-risk reducing effective amount of a misoprostol compound. In some embodiments, the NSAID and the misoprostol compound are co-administered.

In certain embodiments, the methods of reducing NSAID-induced risk of cardiovascular disease, cerebrovascular disease, and/or renovascular disease comprise administering to a subject in need thereof a pharmaceutical composition comprising a core and an outer shell, wherein the core comprises a therapeutically effective amount of a NSAID and a therapeutically effective amount of a misoprostol compound. In some embodiments, the core comprises a first tablet and second tablet, wherein the first tablet comprises the NSAID, and wherein the second tablet comprises the misoprostol compound. In some embodiments, the first tablet is enteric coated.

In certain embodiments, the methods of treating cardiovascular disease, cerebrovascular disease, and/or renovascular disease comprise screening a subject, identifying the subject as at risk for the development or progression of cardiovascular disease, cerebrovascular disease, and/or renovascular disease; and then administering to a subject in need thereof a therapeutically effective amount of a NSAID and a therapeutically effective amount of a misoprostol compound, or a pharmaceutical composition comprising a core and an outer shell, wherein the core comprises a therapeutically effective amount of a NSAID and a therapeutically effective amount of a misoprostol compound.

### DETAILED DESCRIPTION

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Pharmaceutically active: The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” is the pharmaceutically active substance in a drug. As used herein, pharmaceutically active agents include synthetic or naturally occurring small molecule drugs and more complex biological molecules.

Pharmaceutically acceptable: The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

Pharmaceutically acceptable salt: The term “pharmaceutically acceptable salt” as used herein refers to acid addition salts or base addition salts of compounds, such as a NSAID or a misoprostol compound, in the present disclosure. A pharmaceutically acceptable salt is any salt which retains the activity of the parent compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zürich, 2002; Berge et al., J Pharm. Sci. 66: 1, 1977). In some embodiments, a “pharmaceutically acceptable salt” is intended to mean a salt of a free acid or base of a compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

Pharmaceutically acceptable carrier: The terms “pharmaceutically acceptable carrier” as used herein refers to an excipient, diluent, preservative, solubilizer, emulsifier, adjuvant, and/or vehicle with which a compound, such as an NSAID or a misoprostol compound, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

“Treating” or “treatment” or “alleviation” refers to therapeutic treatment wherein the object is to slow down (lessen) if not cure the targeted pathologic condition or disorder or prevent recurrence of the condition. “Treating” or “treatment” or “alleviation” also refers to preventative treatment wherein the treatment is meant to result in a postponement of development of a disease, a symptom of a disease, or medical condition, suppressing symptoms that may appear, or reducing the risk of developing or recurrence of a disease or symptom including a drug-induced adverse event including, but not restricted to, cardiovascular, cerebrovascular or renovascular adverse event. “Treating” or “treatment” or “alleviation” also refers to curative treatments that include reducing the severity of or suppressing the worsening of an existing disease, symptom, or condition. A subject can be considered successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient can also be considered treated if the patient experiences stable disease. A patient can also be considered treated if the onset of a disease is delayed or if the patient is at a lower risk for a drug-induced adverse event including, but not restricted to, cardiovascular, cerebrovascular or renovascular adverse event. In some embodiments, treatment with a therapeutic agent is effective to result in the patients being disease or symptom free, or have a reduced risk of a disease or symptom, 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition such as a drug-induced adverse event. More specific embodiments are included in the sections below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as an infection or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

The term “combination” refers to either a fixed combination in one dosage unit form, or a kit of parts for the combined administration where a compound and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. In certain embodiments, the two active ingredients can be administered simultaneously, within one hour, within 2, 4, 6, 8, 12, 18, or 24 hours of each other. The invention also contemplates a cocktail therapy, e.g., the administration of three or more active ingredients.

It is understood that aspects and embodiments of the compositions of the invention described herein include “consisting” and/or “consisting essentially of” aspects and embodiments, such that other pharmaceutically effective ingredients besides an NSAID and a misoprostol compound are excluded.

In certain embodiments, the following dosages of a misoprostol compound such as misoprostol are useful: 100 ng-10 mg, 1-1 mg, 10 μg-500 μg, and 25 μg-200 μg.

In some embodiments, a therapeutically effective amount of an NSAID is administered to a subject in a therapeutically effective dosing regimen. A therapeutically effective amount of a given NSAID, and its therapeutically effective dosing regimen, are well known to those of ordinary skill in the art.

In certain embodiments, the following daily dosages of the exemplary NSAID diclofenac are useful: 100 μg-1 g, 1 mg-500 mg, and 15 mg-125 mg. In certain embodiments, the following daily dosages of the exemplary NSAID ibuprofen are useful: 100 μg-1 g, 100 mg-2,500 mg, and 600 mg-1,800 mg.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, a subject in need refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

Other objects, advantages and features of the present invention will become apparent from the following specification.

In some aspects, the disclosure provides a unique risk reduction treatment for all individuals taking NSAIDS, regardless of the presence or absence of any specific risk factor any one individual may carry. In some aspects, the disclosure provides a preventative combination treatment that lowers the population-level risk for cardiovascular, cerebrovascular, and/or renovascular adverse events for all persons taking NSAIDs relative to persons taking an NSAID alone.

This disclosure generally provides methods for reducing the risk of NSAID-induced cardiovascular disease, cerebrovascular disease, and/or renovascular disease in a subject by administering a NSAID and a misoprostol compound. The present methods can be used to treat cardiovascular disease, cerebrovascular disease, and/or renovascular disease in any suitable subject. In some embodiments, the subject is a mammal. In some embodiments, the mammal is a human. In other embodiments, the mammal is a non-human mammal, including a pet, a farm animal, an economic animal, a sport animal and an experimental animal, such as a cat, a dog, a horse, a cow, an ox, a pig, a donkey, a sheep, a lamb, a goat, a mouse, a rabbit, a primate, including a monkey and a chimpanzee. In some embodiments, the NSAID and the misoprostol compound are administered separately. In some embodiments, the NSAID and the misoprostol compound are coadministered.

In some embodiments, the NSAID is a salicylate, a propionic acid derivative, an acetic acid derivative, an enolic acid derivative, an anthranilic acid derivative, a selective COX-2 inhibitor, or another nonsteroidal drug with anti-inflammatory properties. In some embodiments, the NSAID can be, but is not limited to, an aceclofenac, a diclofenac, a difluinsal, a febufen, a flufenamic acid, an ibuprofen, an indomethacin, a ketoprofen, a meclofenamate sodium, a meloxicam, a mefenamic acid, a nabumetone, a naproxen, a piroxicam, a suprofen, a tiaprofenic acid, a flurbiprofen, a ketorolac, an oxaprozin, a sulindac, a valdecoxib, a celecoxib, a rofecoxib, or a etoricoxib. In some embodiments, the NSAID can be, but is not limited to, an aspirin (acetylsalicylic acid), a salicylic acid or other salicylate, a salsalate, a pranoprofen, a niflumic acid, a zomepirac, an orpanoxin, an alcofenac, a flenclofenac, a bromfenac, an amfenac, a fenclozic acid, an etodolic acid, a fenbufen, an isofezolac, a benoxaprofen, a fenclorac, a clidanac, a priprofen, an indoprofen, a loxoprofen, a diflunisal, a tolfenamic acid, a tolmetin, an oxaprofen, or a fenoprofen. The NSAID can be any pharmaceutically acceptable salt. Other NSAIDs are well-known to those of ordinary skill in the art.

In some embodiments, a therapeutically effective amount of an NSAID is administered to a subject in a therapeutically effective dosing regimen. A therapeutically effective amount of a given NSAID, and its therapeutically effective dosing regimen, are well known to those of ordinary skill in the art.

As used herein, “misoprostol compound” means misoprostol and any of its metabolites, derivatives, analogs, precursors, and enantiomeric forms. A misoprostol compound can include inorganic and organic salts thereof, pharmaceutically acceptable salts thereof, and organic and inorganic esters of misoprostol, misoprostol acid, and other metabolites, derivatives, analogs, precursors and enantiomorphic forms of misoprostol and misoprostol acid.

This disclosure also generally provides methods for treating cardiovascular disease, cerebrovascular disease, and/or renovascular disease by administering pharmaceutical compositions including a NSAID and a misoprostol compound.

In some embodiments, the methods for treating cardiovascular disease, cerebrovascular disease, and/or renovascular disease comprise orally administering to an NSAID-taking subject a dose of 200 μg misoprostol compound up to four times daily. In some embodiments, the methods for treating cardiovascular disease, cerebrovascular disease, and/or renovascular disease comprise orally administering to an NSAID-taking subject a dose of 100 μg misoprostol compound up to four times daily. The misoprostol compound can be administered with or without food or a liquid.

In some embodiments, the methods for treating cardiovascular disease, cerebrovascular disease, and/or renovascular disease comprise administering to an NSAID-taking subject about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250 μg misoprostol compound per dose.

In some embodiments, the methods for treating cardiovascular disease, cerebrovascular disease, and/or renovascular disease comprise administering to an NSAID-taking subject a dose of misoprostol compound monthly, weekly, or daily. In some embodiments, the methods for treating cardiovascular disease, cerebrovascular disease, and/or renovascular disease comprise administering to an NSAID-taking subject a dose of misoprostol compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 times per week. In some embodiments, the methods for treating cardiovascular disease, cerebrovascular disease, and/or renovascular disease comprise administering to an NSAID-taking subject a dose of misoprostol compound 1, 2, 3, 4, 5, or 6 times per day. In some embodiments, the methods for treating cardiovascular disease, cerebrovascular disease, and/or renovascular disease comprise administering to an NSAID-taking subject a dose of misoprostol compound any time a dose of an NSAID is taken.

In some embodiments, the methods for treating cardiovascular disease, cerebrovascular disease, and/or renovascular disease comprise screening a NSAID-taking subject, identifying the subject as at an increased risk for the development or progression of cardiovascular disease, cerebrovascular disease, and/or renovascular disease, and then administering a therapeutically effective amount of a misoprostol compound. In some embodiments, the NSAID-taking subject is screened for one or more of the following risk factors for cardiovascular, cerebrovascular, or renovascular adverse events: type 1 or type 2 diabetes mellitus, tobacco use (smoking or chewing), hypertension, hyperlipidemia, left ventricular hypertrophy, coronary artery disease, heart failure, peripheral vascular disease, cerebrovascular disease, renovascular disease, thromboembolic disease, physical inactivity, family history of such events, acute or chronic stress, diet high in saturated fat, social isolation, anxiety and depression, individuals taking oral contraceptives or hormone replacement therapy, sleep apnea, atrial fibrillation, high alcohol consumption, obesity, ethnicity, allergy to prescription drugs, illicit drug use, ureter or kidney obstructions, lupus, and contrast dyes for radiological tests. This list of risk factors is non-exclusive, and other risk factors for cardiovascular disease, cerebrovascular disease, and/or renovascular disease are well known to those of ordinary skill in the art. If the NSAID-taking subject has one or more known or observed risk factors for cardiovascular disease, cerebrovascular disease, and/or renovascular disease, the NSAID-taking subject can be administered a therapeutically effective amount of a misoprostol compound to reduce the risk of a cardiovascular disease, cerebrovascular disease, and/or renovascular disease adverse event.

In some embodiments, the methods for treating cardiovascular disease, cerebrovascular disease, and/or renovascular disease comprise administering to an NSAID-taking subject a therapeutically effective amount of a misoprostol compound regardless of the presence of any cardiovascular disease, cerebrovascular disease, and/or renovascular disease risk factors. In some embodiments, the methods for treating cardiovascular disease, cerebrovascular disease, and/or renovascular disease comprise administering to an NSAID-taking subject a therapeutically effective amount of a misoprostol compound when the subject has no risk factors for cardiovascular disease, cerebrovascular disease, and/or renovascular disease.

In some embodiments, the methods for treating cardiovascular disease, cerebrovascular disease, and/or renovascular disease comprise administering to an NSAID-taking subject less than 55 years of age a therapeutically effective amount of a misoprostol compound. In some embodiments, the methods for treating cardiovascular disease, cerebrovascular disease, and/or renovascular disease comprise administering to an NSAID-taking subject 55 years of age or more a therapeutically effective amount of a misoprostol compound. In some embodiments, the subject is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 years of age.

In some embodiments, the methods for treating cardiovascular disease, cerebrovascular disease, and/or renovascular disease comprise administering to an NSAID-taking subject having an average seated systolic blood pressure of less than 140 mm Hg and a diastolic blood pressure of less than 90 mm Hg without antihypertensive medication a therapeutically effective amount of a misoprostol compound. In some embodiments, the methods for treating cardiovascular disease, cerebrovascular disease, and/or renovascular disease comprise administering to an NSAID-taking subject having an average seated systolic blood pressure of 140 mm Hg or more and/or a diastolic blood pressure of 90 mm Hg or more without antihypertensive medication a therapeutically effective amount of a misoprostol compound.

In some embodiments, the methods for treating cardiovascular disease, cerebrovascular disease, and/or renovascular disease comprise administering to an NSAID-taking subject who is salt insensitive a therapeutically effective amount of a misoprostol compound. In some embodiments, the methods for treating cardiovascular disease, cerebrovascular disease, and/or renovascular disease comprise administering to an

NSAID-taking subject who is salt sensitive a therapeutically effective amount of a misoprostol compound. Salt sensitivity is defined by a difference in systolic or diastolic blood pressure of 10 mm Hg or greater between a high and low sodium diet.⁵⁷

In one embodiment, salt sensitivity can be assessed by having a human patient undergo subsequent 10-day periods of high-sodium intake (200 mEq/day) and low-sodium intake (20 mEq/day), separated by the patient's normal dietary sodium intake (˜100 mEq/day) for 1 week. To achieve the high or low-sodium intake, a patient receives 10 identical capsules each day that contain either sodium chloride 20 mEq or lactose in a single-blinded fashion. The high sodium intake precedes the low intake period. A patient can be counseled by a registered dietician to maintain a consistent intake of low sodium-containing foods throughout the salt sensitivity assessment period. Blood pressure is determined by three seated auscultatory blood pressure measurements averaged at the beginning and end of each period. The blood pressure taken at the end of the high-sodium intake period establishes the baseline for the high-sodium period, and the measurement at the end of the normal-sodium diet period is the baseline for the low-sodium period. A patient is classified as salt sensitive if a change in systolic or diastolic blood pressure of 10 mm Hg or greater is detected between the high- and low-sodium periods. If the change is less than 10 mm Hg, the patient is classified as salt insensitive. ⁵⁷

In some embodiments, the methods for treating cardiovascular disease, cerebrovascular disease, and/or renovascular disease reduce the risk of a transient ischemic attack or a cerebrovascular accident. In some embodiments, the methods for treating cardiovascular disease, cerebrovascular disease, and/or renovascular disease reduces the risk of an acute myocardial infarction, an acute coronary syndrome event, or an arrhythmic event. In some embodiments, the methods for treating cardiovascular disease, cerebrovascular disease, and/or renovascular disease reduces the risk of a renovascular adverse event.

The pharmaceutical compositions comprising a NSAID and a misoprostol compound, alone or in combination with other active ingredient(s), described herein may further comprise one or more pharmaceutically-acceptable excipients. A pharmaceutically-acceptable excipient is a substance that is non-toxic and otherwise biologically suitable for administration to a subject. Such excipients facilitate administration of the agent that treats cardiovascular, cerebrovascular, and/or renovascular disease, alone or in combination with other active ingredient(s), described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the various embodiments are sterile compositions. Pharmaceutical compositions may be prepared using compounding techniques known or that become available to those skilled in the art.

Sterile compositions are within the present disclosure, including compositions that are in accord with national and local regulations governing such compositions.

The pharmaceutical compositions, the NSAID, and the misoprostol compound, alone or in combination with other active ingredient(s), described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. The NSAID and the misoprostol compound, alone or in combination with other active ingredient(s), described herein, and preferably in the form of a pharmaceutical composition, may be administered by a suitable route of delivery, such as oral, parenteral, rectal, nasal, topical, or ocular routes, or by inhalation. In some embodiments, the compositions are formulated for intravenous or oral administration.

For oral administration, the NSAID and the misoprostol compound, alone or in combination with another active ingredient, may be provided in a solid form, such as a tablet or capsule, or as a solution, emulsion, or suspension. To prepare the oral compositions, the NSAID and the misoprostol compund, alone or in combination with other active ingredient(s), may be formulated to yield an oral dosage form, such as a tablet, which may include the active ingredient(s) mixed with compatible pharmaceutically acceptable excipients such as diluents, disintegrating agents, binding agents, lubricating agents, sweetening agents, flavoring agents, coloring agents and preservative agents.

Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions may be formulated for rectal administration as a suppository. For parenteral use, including intravenous, intramuscular, intraperitoneal, intranasal, or subcutaneous routes, the agent that treats cardiovascular, cerebrovascular, and/or renovascular disease, alone or in combination with other active ingredient(s), may be provided in sterile aqueous solutions or suspensions, buffered to an appropriate pH and isotonicity or in parenterally acceptable oil. Suitable aqueous vehicles can include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation.

For nasal, inhaled, or oral administration, the NSAID and the misoprostol compound, alone or in combination with other active ingredient(s), may be administered using, for example, a spray formulation also containing a suitable carrier.

For topical applications, the NSAID and the misoprostol compound, alone or in combination with other active ingredient(s), are preferably formulated as creams or ointments or a similar vehicle suitable for topical administration. For topical administration, the NSAID and the misoprostol compound, alone or in combination with other active ingredient(s), may be mixed with a pharmaceutical carrier at a concentration of about 0.1% to about 10% of drug to vehicle. Another mode of administering the NSAID and the misoprostol compound, alone or in combination with other active ingredient(s), may utilize a patch formulation to effect transdermal delivery.

One of ordinary skill in the art may modify the formulations within the teachings of the specification to provide numerous formulations for a particular route of administration. In particular, the NSAID and the misoprostol compound, alone or in combination with other active ingredient(s), may be modified to render them more soluble in water or other vehicle. It is also well within the ordinary skill of the art to modify the route of administration and dosage regimen of the NSAID and the misoprostol compound, alone or in combination with other active ingredient(s), in order to manage the pharmacokinetics of the present compounds for maximum beneficial effect in a patient.

### EXAMPLES

In a historical study using regression modeling with matched weighting of patient records from the Veterans Affairs Informatics and Computing Infrastructure Data Warehouse, patients who were new initiators of misoprostol or NSAIDs from Jan. 1, 2005 to Dec. 31, 2013 were followed for 5 years or end of the observation year for cardiovascular adverse events (acute myocardial infarction, cardiac arrest, or ventricular fibrillation), cerebrovascular adverse events (acute cerebrovascular disease, occlusion or stenosis of pre-cerebral arteries, transient cerebral ischemia, or other ill-defined cerebrovascular disease) or renovascular adverse events (acute and unspecified renal failure). The records of patients (aged≥18 years of age) were included in the study if they were prescribed NSAIDS alone or in free combination with misoprostol for ≥3 months. Count outcomes of cardiovascular, cerebrovascular, or renovascular events were assessed with incident rate ratios (IRR) which were estimated using weighted bivariate negative binomial regression models.

Patients who initiated NSAIDs were 1,338,547 and 4,737 initiated misoprostol with 43.0% using NSAIDs and 42.4% using misoprostol for >3 months, respectively. The final groups for cardiovascular, cerebrovascular, or renovascular event determination consisted of 542,624 subjects for NSAID use alone and 1,814 subjects who used NSAIDs and misoprostol. After matched weighting the analyzed cohort was 1814 for NSAID use alone and NSAID and misoprostol, respectively.

Eleven categories of matching variables which included demographics, laboratory values, and disease states and forty-eight pre-treatment baseline covariates were identified as possible cofounders based on clinical judgement and literature review that indicated these variables might influence treatment decisions and persistency at one year of follow-up. Using a multivariate logistic regression model, with treatment group as the outcome and the list of possible confounders as the predictors, a propensity score was generated. The propensity score was then included in a Poisson regression model using the method of matching weights. (Li and Greene 2013).

The mean age was 62.5±10.4 years of age for the NSAID use alone group and 67.4±11.3 for the NSAID plus misoprostol group. The majority of the total study population (NSAID and NSAID plus misoprostol groups) were male and non-white, had hypertension and hyperlipidemia, with 30% diagnosed with diabetes and 16-24% having coronary atherosclerosis and other heart disease.

The misoprostol cohort prior to matching were significantly older with significantly higher rates of coronary atherosclerosis, cardiac arrhythmias, congestive heart failure, chronic kidney disease, and hypertension. The Charlson Score was 0.92±1.35 (μ±SD) for NSAID cohort versus 1.1±1.4 for misoprostol, p<0.001. After weighting, there were no statistical differences for any characteristic between the cohorts.

The matching weight adjusted model showed statistically significant reductions in cardiovascular, cerebrovascular and renovascular endpoints in the NSAID plus misoprostol cohort compared to the NSAID use alone cohort. Table 1 shows the IRR for the likelihood each adverse event including the statistical significance and percent reduction. Specifically, cardiovascular endpoints were significantly reduced with NSAID plus misoprostol with an IRR of 0.72 (95% CI: 0.71-0.73).

Cerebrovascular endpoints were significantly reduced with NSAID plus misoprostol with an IRR of 0.82 (95% CI: 0.82-0.83) (Table 1).

Renovascular endpoints were significantly reduced with NSAID plus misoprostol with an IRR of 0.64 (95% CI: 0.64-0.66).

TABLE 1

| Adverse Events: | Cardiovascular | Cerebrovascular | Renovascular |
|---|---|---|---|
| NSAID Alone* | 52.1 | 221.0 | 145.9 |
| NSAID + | 37.5 | 181.0 | 95.5 |
| Misoprostol* | | | |
| IRR | 0.72 (0.71- | 0.82 (0.82- | 0.64 (0.64- |
| | 0.73) | 0.83) | 0.65) |
| P-Value | <0.0001 | <0.0001 | <0.0001 |
| % Reduction | −28% | −18% | −35% |

*Rate per 100,000 persons

### REFERENCES

1. Vane J R, Bakhle Y S, Botting R M. Annu Rev Pharm Toxicol 1998; 38:97-120.
2. Wiegand T J, et al., Nonsteroidal Anti-inflammatory Agent Toxicity, Medscape (2012), available at http://emedicine.medscape.com/article/816117-overview#a5
3. McGettigan P, Henry D. Use of non-steroidal anti-inflammatory drugs that elevate cardiovascular risk: An examination of sales and essential medicine lists in low-, middle-, and high-income countries. PLOS Medicine 2013; 10(2):e1001388
4. Chou R, Helfand M, Peterson K, Dana T, Roberts C. (2006) Comparative effectiveness and safety of analgesics for osteoarthritis: comparative effectiveness review: Agency for Healthcare Research and Quality. http://effectivehealthcare.ahrq.gov/repFiles/AnalgesicsFinal.pdf Accessed September 2015
5. Dajani E Z. Gastrointestinal toxicity of over-the-counter analgesics. Am J Gastroenterol 1998; 93(7):1020-2.
6. Griffin M R, Piper J M, Daugherty J R, et al. Non-steroidal anti-inflammatory drug use and increased risk for peptic ulcer disease in elderly persons. Ann Intern Med 1991; 114(4):257-63.
7. Laine L. Nonsteroidal anti-inflammatory drug gastropathy. Gastroinest Endosc Clin N Am 1996; 6:489-504.
8. Bombardier C, Laine L, Reicin A, Shapiro D, Vurgos-Vargas R, et al. Comparison of upper respiratory gastrointestinal toxicity of rofecoxib and naproxen in patients with rheumatoid arthritis. N Engl J Med 2000; 343:1520-28.
9. Blot W J, McLaughlin J K. Over the counter non-steroidal anti-inflammatory drugs and risk of gastrointestinal bleeding. J Epidemiol Biostat 2000; 5(2):137-42.
10. Bertagnolli M, Eagle C J, Zauber A G, Redston M, Solomon S D, et al. Celecoxib for the prevention of sporadic colorectal ademonmas. N Engl J Med 2006; 355:873-884.
11. Pirmohamed M, James S, Meakin, et al. Adverse drug reactions as cause of admission to hospital: prospective analysis of 18,820 patients. BMJ 2004; 329:15-19.
12. McGettigan P, Henry D. Cardiovascular risk with non-steroidal anti-inflammatory drugs: systematic review of population-based controlled observational studies. PLOSMedicine 2011; 8(9):1-18.
13. FDA News Release, FDA Issues Public Health Advisory on Vioxx as its Manufacturer Voluntarily Withdraws the Product, Sep. 30, 2004, available at http://www.fda.gov/NewsEvents/Newsroom/PressAnnouncements/2004/ucm108361.ht
14. Bextra (valdecoxib), April 2005, available at http://www.fda.gov/Safety/MedWatch/SafetyInformation/SafetyAlertsforHumanMedicalProducts/ucm150752.htm
15. FDA Drug Safety Communication: FDA strengthens warning that non-aspirin nonsteroidal anti-inflammatory drugs (NSAIDs) can cause heart attacks or strokes, Jul. 9, 2015, available at http://www.fda.gov/Drugs/DrugSafety/ucm451800.htm
16. Gislason G H, Rasmussen J N, Abildstrom S Z, et al. Increased mortality and cardiovascular mortality associated with anti-inflammatory drugs in chronic heart failure. Arch Intern Med 2009; 169(2):141-9.
17. Mamdani M, Juurlink D N, Lee D S, et al. Cyclo-osygenase-2 inhibitors versus non-selective non-steroidal anti-inflammatory drugs and congestive heart failure outcomes in elderly patients: a population-based cohort study. Lancet 2004; 363:1751-6.
18. Francois H, Athirakul K, Howell D et al. Prostacyclin protects against elevated blood pressure and cardiac fibrosis. Cell Metab 2005; 2:201-7.
19. Watanabe H, Katoh T, Eiro M, Iwanmato M, Ushikubi F, Narumiya S, Watanabe T. Effects of salt loading on blood pressure in mice lacking the prostanoid receptor gene. Circ J 2005; 69(1):124-6.
20. Rudic R D, Brinster D, Cheng Y, Fries S, Song W L, Austin S, Coffman T M, Fitzgerald G A. Cox-2-derived prostacyclin modulates vascular remodeling. Circ Res 2005; 24:96(12):1240-7.
21. Fitzgerald G A. Coxibs and cardiovascular disease. N Engl J Med 2004; 351:1709-11.
22. Strand V, Hochberg M C. The risk of cardiovascular thrombotic events with selective cyclooxygenase-2 inhibitors. Arthritis Rheum 2002:47:349-55.
23. Kobayashi T, Tahara Y, Matsumoto M, et al. Roles of thromboxane A2 and prostacyclin in the development of atherosclerosis in apoE-deficient mice. J Clin Invest 2004; 114:784-94.
24. Egan K M, Lawson J A, Fries S, Koller B, Rader D J, Smyth E M, Fitzgerald G A. COX-2-derived prostacyclin confers atheroprotection on female mice. Science 2004; 306:1954-7.
25. Schneider V, Levesque L E, Zhang B, et al. Association of selective and conventional nonsteroidal antiinflammatory drugs with acute renal failure: A population, nested case-control analysis. Am J Epidemiol 2006; 164:881.
26. Swan S K, Rudy D W, Lasseter K C, et al. Effect of cyclooxygenase-2 inhibition on renal function in elderly persons receiving a low-salt diet. A randomized, controlled trial. Ann Intern Med 2000; 133:1-9.
27. Lapi F, Azoulay L, Yin H., et al. Concurrent use of diuretics, angiotensin converting enzyme inhibitors, and angiotensin receptor blockers with non-steroidal anti-inflammatory drugs and risk of acute kidney injury: nested case-control study. BMJ 2013; 346:e8525
28. Wei L, MacDonald T M, Jennings C, et al. Estimated GFR reporting is associated with decreased nonsteroidal anti-inflammatory drug prescribing and increased renal function. Kidney Int 2013; 84:174-8.
29. Huerta C, Castellsague J, Varas-Lorenzo C, Garcia Rodriguez L A. Nonsteroidal anti-inflammatory drugs and risk of ARF in the general population. Am J Kidney Dis 2005; 45:531-9.
30. Griffin M R, Yared A, Ray W A. Nonsteroidal anti-inflammatory drugs and acute renal failure in elderly persons. Am J Epidemiol 2000; 15(5):488-96.
31. Plantinga L, Grubbs V, Sarkar U, et al. Nonsteroidal anti-inflammatory drug use among persons with chronic kidney disease in the United States. Ann Fam Med 2011; 9:423-30.
32. Chou S Y, Dahhan A, Porush J G. Renal actions of endothelin: interaction with prostacyclin. Am J Physiol 1990; 259:F645.
33. Oliver J A, Pinto J, Sciacca R R, Cannon P J. Increased renal section of norepinephrine and prostaglandin E2 during sodium depletion in the dog. J Clin Invest 1980; 66(4):748-56.
34. Scharschmidt L A, Dunn M J. Prostaglandin synthesis by rat glomerular mesangial cells in culture. Effects of angiotensin II and arginine vasopressin. J Clin Invest 1983; 71:1756-64.
35. Yared A, Kon V, Ichikawa I. Mechanism of preservation of glomerular perfusion and filtration during acute extracellular fluid volume depletion. Importance of intrarenal vasopressin-prostaglandin interaction for protecting kidneys from constrictor action of vasopressin. J Clin Invest 1985; 75:1477-87.
36. Freeman R H, Davis J O, Villarreal D. The role of prostaglandins in the control of renin release. Circ Res 1984; 54:1-9.
37. Ito S, Carretero O A, Abe K, et al. Effect of prostanoids on renin release from rabbit afferent arterioles with and without macula densa. Kidney Int 1989; 35:1138-44.
38. Hebert R L, Jacobson H R, Breyer M D. PGE2 inhibits AVP-induced water flow in cortical collecting ducts by protein kinase C activation. Am J Physiol 1990; 259:F318.
39. Steiner J A. Misoprostol clinical pharmacology. Establishment of activity in man Digest Disease Sci 1985; 30(11):136S-41S.
40. Hayashida M, Hanaoka K, Shimada Y, Namiki A, Amaha K. The effect of low-dose prostaglandin E1 on circulation, respiration, and body temperature during surgical anesthesia. Masui 1987; 46(3):363-72.
41. Hayashida M, Hanaoka K, Shimada Y, Namiki A, Amaha K. The effect of low-dose prostaglandin E1 on intra- and post-operative renal function. Masui 1997; 46(4):464-70.
42. Bakris G L. Renal effects of oral prostaglandin supplementation after ibuprofen in diabetic subjects: a double-blind, placebo-controlled, multicenter trial. J Am Soc Nephrol 1995; 5:1684-8.
43. Pfizer, “Cytotec® misoprostol tablets,” available at http://www.accessdata.fda.gov/drugsatfda_docs/label/2009/019268s0411bl.pdf.
44. Munger M A, Gardner S F, Ateshkadi, et al. Misoprostol effects on diclofenac-induced cardiorenal changes in salt-sensitive patients with hypertension: The MEDIC Study. Pharmacother 2008; 28(7):834-42.
45. World Heart Federation, “Stroke,” available at http://www.world-heart-federation.org/cardiovascular-health/stroke/.
46. National Health Service, “Cerebrovascular disease—Risks and prevention,” available at http://www.nhs.uk/Conditions/Cerebrovascular-disease/Pages/Prevention.aspx.
47. Life Options, “Risk Factors for CKD,” available at http://lifeoptions.org/kidneyinfo/ckdinfo.php?page=2.
48. Centers for Disease Control, “Summary Health Statistics for U.S.Adults: National Health Interview Survey, 2012” available at http://www.cdc.gov/nchs/data/series/sr_10/sr10_260.pdf.
49. Li J, et al. Neuroscience Letters 2008; 438:210-15
50. Cooper DL et al. PLOSone 2013; 9(2):e89097.
51. Ray J G. Transplantation 1998; 66(4):476-83.
52. Davies C, et al. Am J Vet Res 1998; 59(8):1048-54.
53. Hsia H C, et al. Scand J Gastroenterol 1995; 30(12):1194-8.
54. Weinblatt M E, et al. Arthritis Rheum 1994; 37(9):1321-5.
55. Rudy D W, et al. Am J Ther 1995; 2(11):864-74.
56. Whelton A, et al. Am J Ther 1995; 2(11):858-63.
57. Ferri C, Bellini C, Picolli A, et al Enhanced blood pressure to cyclooxygenase inhibition in salt-sensitive essential hypertension. Hypertension 1993; 21:875-81.

1. A method of reducing NSAID-induced risk of cardiovascular, cerebrovascular, and/or renovascular adverse events in a subject comprising:
administering to a subject in need thereof a therapeutically effective amount of a NSAID and a therapeutically effective amount of a misoprostol compound, thereby reducing a NSAID-induced risk of cardiovascular, cerebrovascular, or renovascular adverse events, or a combination thereof, in the subject.

2. The method of claim 1, wherein the NSAID and the misoprostol compound are coadministered.

3. A method of reducing NSAID-induced risk of cardiovascular, cerebrovascular, and/or renovascular adverse events in a subject comprising:
administering to a subject in need thereof a pharmaceutical composition comprising a core and an outer shell, wherein the core comprises a therapeutically effective amount of a NSAID and a therapeutically effective amount of a misoprostol compound, thereby reducing a NSAID-induced risk of cardiovascular, cerebrovascular, or renovascular adverse events, or a combination thereof, in the subject.

4. The method of claim 3, wherein the core comprises a first tablet and second tablet, wherein the first tablet comprises the NSAID, and wherein the second tablet comprises the misoprostol compound.

5. The method of claim 4, wherein the first tablet is enteric coated.

6. The method of claim 1, wherein the NSAID is selected from an aceclofenac, a diclofenac, a difluinsal, a febufen, a flufenamic acid, an ibuprofen, an indomethacin, a ketoprofen, a meclofenamate sodium, a meloxicam, a mefenamic acid, a nabumetone, a naproxen, a piroxicam, a suprofen, a tiaprofenic acid, a flurbiprofen, a ketorolac, an oxaprozin, a sulindac, a valdecoxib, a celecoxib, a rofecoxib, a etoricoxib, and combinations thereof.

7. The method of claim 1, wherein the subject in need thereof is a human.

8. The method of claim 7, wherein the human is less than 55 years of age.

9. The method of claim 7, wherein the human has an average seated systolic blood pressure of less than 140 mm Hg and a diastolic blood pressure of less than 90 mm Hg without antihypertensive medication.

10. The method of claim 7, wherein the human is salt insensitive.

11. The method of claim 1, wherein the step of administering the NSAID and the misoprostol compound reduces the risk of a cardiovascular adverse event

12. The method of claim 1, wherein the step of administering the NSAID and the misoprostol compound reduces the risk of a cerebrovascular adverse event

13. The method of claim 1, wherein the step of administering the NSAID and the misoprostol compound reduces the risk of a renovascular adverse event.

14. The method of claim 1 further comprising:
screening the subject;
identifying the subject as at risk for the development or progression of cardiovascular, cerebrovascular, renovascular adverse events, or a combination thereof; and then
adminstering the NSAID and the misoprostol compound.

15. The method of claim 1, wherein the therapeutically effective amount of misoprostol compound is about 25 μg to about 200 μg misoprostol compound per dose.

16. The method of claim 1, wherein the misoprostol compound is misoprostol.

17. The method of claim 1, wherein the NSAID and the misoprostol compound are administered simultaneously.

18. The method of claim 1, wherein the NSAID and the misoprostol compound are administered within 0 to 24 hours of each other.

19. A method of reducing NSAID-induced cardiovascular, cerebrovascular, and/or renovascular adverse events in a subject comprising:
administering to a NSAID-taking subject in need thereof a therapeutically effective amount of a misoprostol compound, thereby reducing a NSAID-induced risk of cardiovascular, cerebrovascular, or renovascular adverse events, or a combination thereof, in the subject.

20. The method of claim 19, wherein the subject in need thereof is a human.

21. The method of claim 19, wherein the therapeutically effective amount of misoprostol compound is about 25 μg to about 200 μg misoprostol compound per dose.

22. The method of claim 19, wherein the misoprostol compound is misoprostol.

23. The method of claim 19, wherein the misoprostol compound is administered within 0 to 24 hours of the subject having taken a NSAID.
